(19) United States
(12) Patent Application Publication
(10) Pub. No.: US 2011/0034783 A1
(43) Pub. Date: Feb. 10, 2011

(54) SYSTEMS AND METHODS FOR BALANCING POWER CONSUMPTION AND UTILITY OF WIRELESS MEDICAL SENSORS

(75) Inventors: Daniel Lisogurski, Boulder, CO (US); Edward M. McKenna, Boulder, CO (US)

Correspondence Address: NELLCOR PURITAN BENNETT LLC;ATTN: IP LEGAL, 6135 Gunbarrel Avenue, Boulder, CO, 80301, US

(73) Assignee: Nellcor Puritan Bennett LLC, Boulder, CO (US)

(21) Appl. No.: 12/538,696

(22) Filed: Aug. 10, 2009

(51) Int. Cl. A61B 5/00

(52) U.S. Cl. 600/301

(57) ABSTRACT

Systems, methods, and devices for balancing power consumption and utility of medical sensors are provided. For example, a wireless medical sensor device may include a sensor, data processing circuitry, and wireless transmission circuitry. The sensor may be capable of obtaining a raw measurement from a patient, and the data processing circuitry may be capable of sampling the raw measurement to obtain values. Further, the data processing circuitry also may be capable of determining an update interval based at least in part on an update factor associated with a status of the patient, and the wireless transmission circuitry may be capable of wirelessly transmitting one of the values to an external wireless receiver at the update interval.

### BACKGROUND

The present disclosure relates generally to medical sensors and, more particularly, to wireless medical sensors.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

Medical sensors are used in a variety of medical applications. For example, a plethysmographic sensor may provide such information as patient pulse rate, blood oxygen saturation, and/or total hemoglobin, or a respiration band may provide the respiration rate of a patient. Such medical sensors may communicate with a local patient monitor or a network using a communication cable. However, the use of communication cables may limit the range of applications available, as the cables may become prohibitively expensive at long distances and may physically tether a patient to a monitoring device, limiting patient range of motion. Though wireless medical sensors may transmit information without need of a communication cable, wireless medical sensors may employ large batteries that are cumbersome, uncomfortable to wear, and expensive.

### SUMMARY

Certain aspects commensurate in scope with the originally claimed embodiments are set forth below. It should be understood that these aspects are presented merely to provide the reader with a brief summary of certain forms the embodiments might take and that these aspects are not intended to limit the scope of the presently disclosed subject matter. Indeed, the embodiments may encompass a variety of aspects that may not be set forth below.

Present embodiments relate to systems, methods, and devices for balancing power consumption and utility of medical sensors. For example, a wireless medical sensor device may include a sensor, data processing circuitry, and wireless transmission circuitry. The sensor may be capable of obtaining a raw measurement from a patient, and the data processing circuitry may be capable of sampling the raw measurement to obtain discrete values. Further, the data processing circuitry also may be capable of determining an update interval based at least in part on a predetermined update factor associated with a status of the patient, and the wireless transmission circuitry may be capable of wirelessly transmitting one of the discrete values to an external wireless receiver at the update interval.

### BRIEF DESCRIPTION OF THE DRAWINGS

Advantages of the presently disclosed subject matter may become apparent upon reading the following detailed description and upon reference to the drawings in which:

FIG. 1 is a perspective view of a wireless medical sensor system, in accordance with an embodiment;

FIG. 2 is a block diagram of the system of FIG. 1, in accordance with an embodiment;

FIG. 3 is a flowchart describing an embodiment of a method for providing wireless medical sensor data using the system of FIG. 1, in accordance with an embodiment;

FIG. 4 is a schematic diagram of various factors that may be employed with the method of FIG. 3, in accordance with an embodiment;

FIG. 5 is a communication diagram schematically illustrating communication between a wireless medical sensor and a patient monitor of the system of FIG. 1, in accordance with an embodiment;

FIG. 6 is another communication diagram schematically illustrating communication between the wireless medical sensor and the patient monitor of the system of FIG. 1, in accordance with an embodiment;

FIG. 7 is a schematic diagram of parameters for controlling the system of FIG. 1, in accordance with an embodiment;

FIG. 8 is a flowchart describing an embodiment of a method for transmitting wireless sensor data at a context-based latency, in accordance with an embodiment;

FIG. 9 is a communication diagram illustrating communication between the wireless sensor and the patient monitor of the system of FIG.1 while carrying out the method of FIG. 8, in accordance with an embodiment; and

FIG. 10 is a flowchart of an embodiment of a method for wirelessly transmitting medical data at a discrete context-based data transfer level, in accordance with an embodiment.

### DETAILED DESCRIPTION OF SPECIFIC EMBODIMENTS

One or more specific embodiments of the present disclosure will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Present embodiments may apply to a variety of wireless medical sensors, including photoplethysmographic sensors, temperature sensors, respiration bands, blood pressure sensors, ECG sensors, pulse transit time sensors, and so forth. Moreover, as disclosed herein, the particular data of interest that may be observed using a wireless medical sensor may similarly vary depending on the capabilities of each device. For example, a photoplethysmographic sensor may transmit data of interest that includes pulse rate, blood oxygen saturation, and/or total hemoglobin, and so forth. Because the embodiments presently disclosed may reduce the quantity of data to be transmitted wirelessly, the wireless medical sensors may expend less power and, accordingly, may employ smaller or less expensive batteries, which may be more comfortable to wear

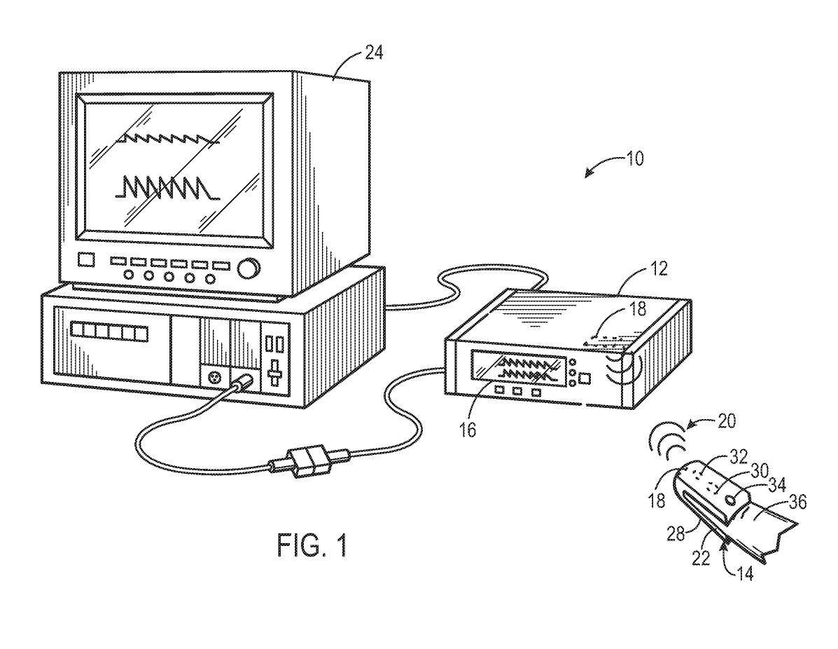

With the foregoing in mind, FIG. 1 illustrates a perspective view of an embodiment of a wireless medical sensor system 10 that may efficiently transmit and/or receive medical sensor data, conserving power. Although the embodiment of the system 10 illustrated in FIG. 1 relates to wireless photoplethysmography, the system 10 may be configured to obtain a variety of medical measurements with a suitable medical sensor, For example, the system 10 may, additionally or alternatively, be configured to obtain a respiration rate, a patient temperature, an ECG, a blood pressure, and/or a pulse transit time, and so forth.

The system 10 may include a patient monitor 12 that communicates wirelessly with a wireless medical sensor 14. The patient monitor 12 may include a display 16, a wireless module 18 for transmitting and receiving wireless data, a memory, a processor, and various monitoring and control features. Based on sensor data received from the wireless medical sensor 14, the patient monitor 12 may display patient measurements and perform various additional algorithms. For example, when the system 10 is configured for photoplethysmography, the patient monitor may perform pulse oximetry measurements, calculations, and control algorithms, based on the received wireless sensor data.

In the presently illustrated embodiment of the system 10, the wireless medical sensor 14 is a photoplethysmographic sensor. As should be appreciated, however, the sensor 14 may be chosen to obtain any of a variety of medical measurements, such as a respiration rate, a patient temperature, an ECG, a blood pressure, and/or a pulse transit time, and so forth. Like the patient monitor 12, the sensor 14 may also include a wireless module 18. The wireless module 18 of the sensor 14 may establish wireless communication 20 with the wireless module 18 of the patient monitor 12 using any suitable protocol. By way of example, the wireless modules 18 may be capable of communicating using the IEEE 802.15.4 standard, and may be, for example, ZigBee, WirelessHART, or MiWi modules. Additionally or alternatively, the wireless modules 18 may be capable of communicating using the Bluetooth standard or one or more of the IEEE 802.11 standards. As described further below, the wireless module 18 of the sensor 14 may transmit data of interest at an interval that depends on one or more factors relating to the context of its use. Thus, the wireless module 18 may not consume excessive power while the wireless medical sensor 14 provides medical data about a patient.

A sensor assembly or body 22 of the wireless medical sensor 14 may attach to patient tissue (e.g., a patient's finger, ear, forehead, or toe). In the illustrated embodiment, the sensor assembly 22 is configured to attach to a finger. The system 10 may also include a separate display feature 24 that is communicatively coupled with the patient monitor 12 to facilitate presentation of medical data, such as plethysmographic data. By way of example, the display feature 24 may display a plethysmogram, pulse oximetry information, non-invasive measurement of total hemoglobin, and/or related data.

The wireless medical sensor 14, illustrated in the present embodiment as a photoplethysmographic sensor, may include an emitter 28 and a detector 30. When attached to pulsatile tissue, the emitter 28 may transmit light at certain wavelengths into the tissue and the detector 30 may receive the light after it has passed through or is reflected by the tissue. The amount of light that passes through the tissue and other characteristics of light waves may vary in accordance with the changing amount of certain blood constituents in the tissue and the related light absorption and/or scattering. For example, the system 10 may emit light from two or more LEDs or other suitable light sources into the pulsatile tissue. The reflected or transmitted light may be detected with the detector 30, such as a photodiode or photo-detector, after the light has passed through or has been reflected by the pulsatile tissue.

One or more additional medical sensors may also be present in the sensor 14. In addition to the emitter 28 and the detector 30, the sensor 14 may include an extraneous sensor 32 for monitoring a patient characteristic that may be extraneous to photoplethysmography. By way of example, the extraneous sensor 32 may include a temperature sensor to measure a current temperature at the pulsatile tissue site. This extraneous measurement may be used as a factor in determining a wireless data update rate, as discussed in greater detail below.

A button or switch 34 may enable a patient 36 or medical staff associated with the patient 36 to indicate an operating preference of the wireless medical sensor 14. Such operating preferences may include a level of granularity of the medical data transferred, a request for raw photoplethysmographic data for a predetermined time, a change in the data of interest to be transferred, a preferred wavelength to be employed by the emitter 28, and so forth. In one embodiment, the button or switch 34 may be a button that, when pressed, may instruct the sensor 14 that all raw data is to be transferred to the patient monitor 12. In another embodiment, the button or switch 34 may be a switch with two or more settings to indicate that the data of interest is to be transferred at a discrete data transfer level (e.g., low, medium, or high). The selection of the button or switch 34 may also be used as a factor in determining the wireless data update rate of a measurement or sampling interval of a waveform, as discussed below.

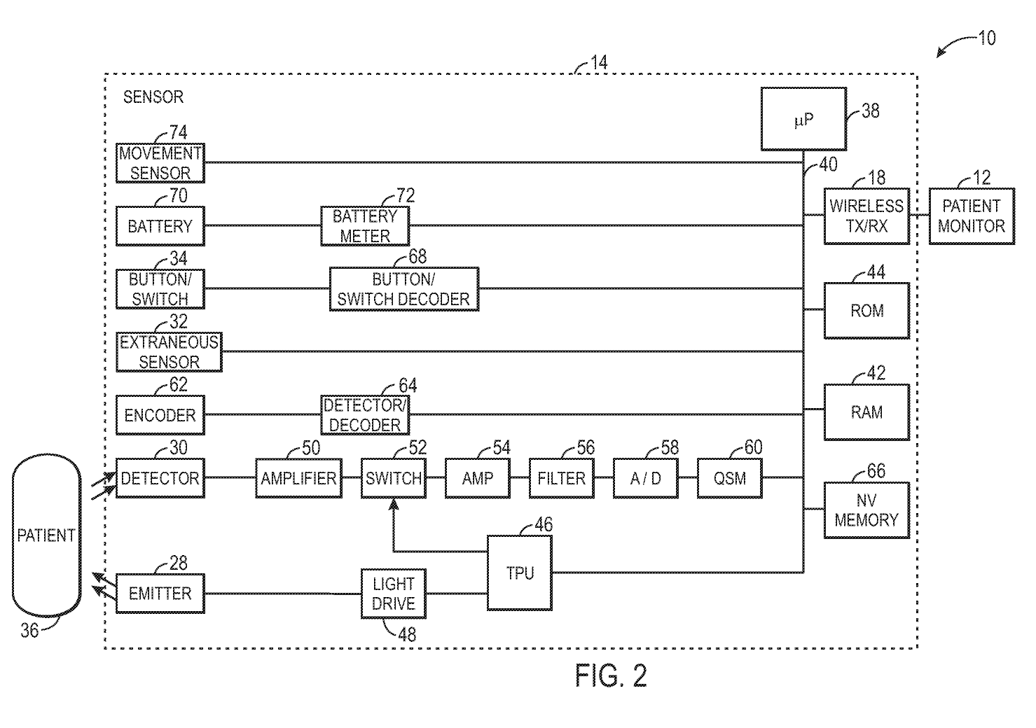

FIG. 2 is a block diagram of an embodiment of the wireless medical sensor system 10 that may be configured to implement the techniques described herein. By way of example, embodiments of the system 10 may be implemented with any suitable medical sensor and patient monitor, such as those available from Nellcor Puritan Bennett LLC. The system 10 may include the patient monitor 12 and the sensor 14, which may be configured to obtain, for example, a plethysmographic signal from patient tissue at certain predetermined wavelengths. The photoplethysmographic sensor 14 may be communicatively connected to the patient monitor 12 via wireless communication 20 (shown in FIG. 1). When the system 10 is operating, light from the emitter 28 may pass into the patient 36 and be scattered and detected by the detector 30. The sensor 14 may include a microprocessor 38 connected to a bus 40. Also connected to the bus 40 may be a RAM memory 42 and an optional ROM memory 44. A time processing unit (TPU) 46 may provide timing control signals to light drive circuitry 48 which may control when the emitter 28 is illuminated, and if multiple light sources are used, the multiplexed timing for the different light sources. The TPU 46 may optionally also control the gating-in of signals from the detector 30 through an amplifier 50 and a switching circuit 52. These signals may be sampled at the proper time, depending upon which of multiple light sources is illuminated, if multiple light sources are used. The received signal from the detector 30 may be passed through an amplifier 54, a low pass filter 56, and an analog-to-digital converter 58.

The digital data may then be stored in a queued serial module (QSM) 60, for later downloading to the RAM 42 as the QSM 60 fills up. Alternatively, the processor 38 may read the A/D converter after each sample, without the use of QSM 60. In one embodiment, there may be multiple parallel paths of separate amplifier, filter and A/D converters for multiple light wavelengths or spectra received. This raw digital data may be further processed by the wireless medical sensor 14 into specific data of interest, such as pulse rate, blood oxygen saturation, and so forth. The data of interest may take up significantly less storage space than the raw data. For example, a raw 16-bit digital stream of photoplethysmographic data of between approximately 50 Hz or less to 2000 Hz or more (e.g., approximately 1211 Hz) may be sampled down to between approximately 10 Hz to 200 Hz (e.g., approximately 57.5 Hz), before being processed to obtain an instantaneous pulse rate at a given time, which may take up only approximately 8 bits.

In an embodiment, the sensor 14 may also contain an encoder 62 that provides signals indicative of the wavelength of one or more light sources of the emitter 28, which may allow for selection of appropriate calibration coefficients for calculating a physiological parameter such as blood oxygen saturation. The encoder 62 may, for instance, be a coded resistor, EEPROM or other coding devices (such as a capacitor, inductor, PROM, RFID, parallel resonant circuits, or a colorimetric indicator) that may provide a signal to the processor 38 related to the characteristics of the photoplethysmographic sensor 14 that may allow the processor 38 to determine the appropriate calibration characteristics for the photoplethysmographic sensor 14. Further, the encoder 62 may include encryption coding that prevents a disposable part of the photoplethysmographic sensor 14 from being recognized by a processor 38 that is not able to decode the encryption. For example, a detector/decoder 64 may be required to translate information from the encoder 62 before it can be properly handled by the processor 38. In some embodiments, the encoder 62 and/or the detector/decoder 64 may not be present. Additionally or alternatively, the processor 38 may encode processed sensor data before transmission of the data to the patient monitor 12.

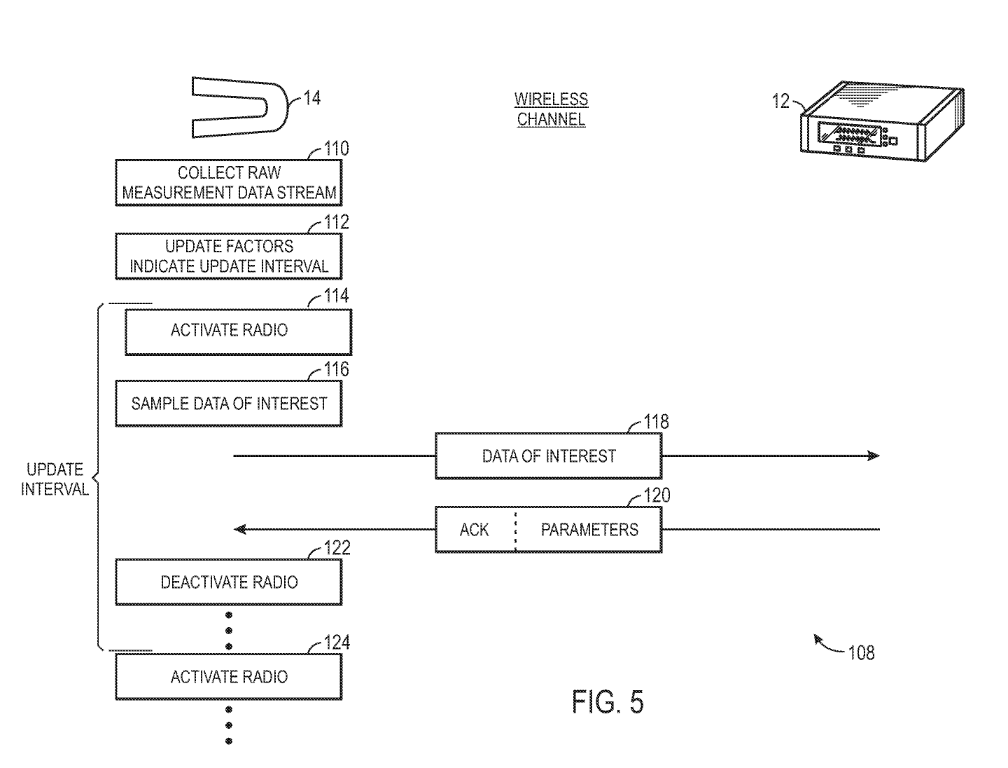

In various embodiments, based at least in part upon the value of the received signals corresponding to the light received by detector 30, the microprocessor 38 may calculate a physiological parameter of interest using various algorithms. These algorithms may utilize coefficients, which may be empirically determined, corresponding to, for example, the wavelengths of light used. These may be stored in the ROM 44 or in other nonvolatile memory 66 including flash or One-Time Programmable (OTP) memory. In a two-wavelength system, the particular set of coefficients chosen for any pair of wavelength spectra may be determined by the value indicated by the encoder 62 corresponding to a particular light source provided by the emitter 28. For example, the first wavelength may be a wavelength that is highly sensitive to small quantities of deoxyhemoglobin in blood, and the second wavelength may be a complimentary wavelength. Specifically, for example, such wavelengths may be produced by orange, red, infrared, green, and/or yellow LEDs. Different wavelengths may be selected based on instructions from the patient monitor 12, based preferences stored in a nonvolatile storage 66, or depending on whether the button or switch 34 has been selected, as determined by the button or switch decoder 68 or automatically based on an algorithm executed by the processor 38. The instructions from the patient monitor 12 may be transmitted wirelessly to the sensor 14 in the manner described below with reference to FIG. 5, and may be selected at the patient monitor 12 by a switch on the patient monitor 12, a keyboard, or a port providing instructions from a remote host computer.

Nonvolatile memory 66 may store caregiver preferences, patient information, or various parameters, discussed below, which may be used in the operation of the sensor 14. Software for performing the configuration of the sensor 14 and for carrying out the techniques described herein may also be stored on the nonvolatile memory 66, or may be stored on the ROM 44. The nonvolatile memory 66 and/or RAM 42 may also store historical values of various discrete medical data points. By way of example, the nonvolatile memory 66 and/or RAM 42 may store values of instantaneous pulse rate for every second or every heart beat of the most recent five minutes. These stored values may be used as factors in determining the wireless data update rate, as discussed in greater detail below.

A battery 70 may supply the wireless medical sensor 14 with operating power. By way of example, the battery 70 may be a rechargeable battery, such as a lithium ion or lithium polymer battery, or may be a single-use battery such as an alkaline or lithium battery. Due to the techniques described herein to reduce battery consumption, the battery 70 may be of a much lower capacity, and accordingly much smaller and/or cheaper, than a battery needed to power a similar wireless sensor that does not employ these techniques. A battery meter 72 may provide the expected remaining power of the battery 70 to the microprocessor 38. The remaining battery life indicated by the battery meter 72 may be used as a factor in determining the wireless data update rate, as discussed in greater detail below.

The wireless medical sensor 14 may also include a movement sensor 74 that may sense when the patient 36 moves the sensor 14. The movement sensor 74 may include, for example, a digital accelerometer that may indicate a state of motion of the patient 36. Whether the patient is at rest or moving, as indicated by the movement sensor 74, may also be used as a factor in determining the wireless data update rate, as discussed in greater detail below.

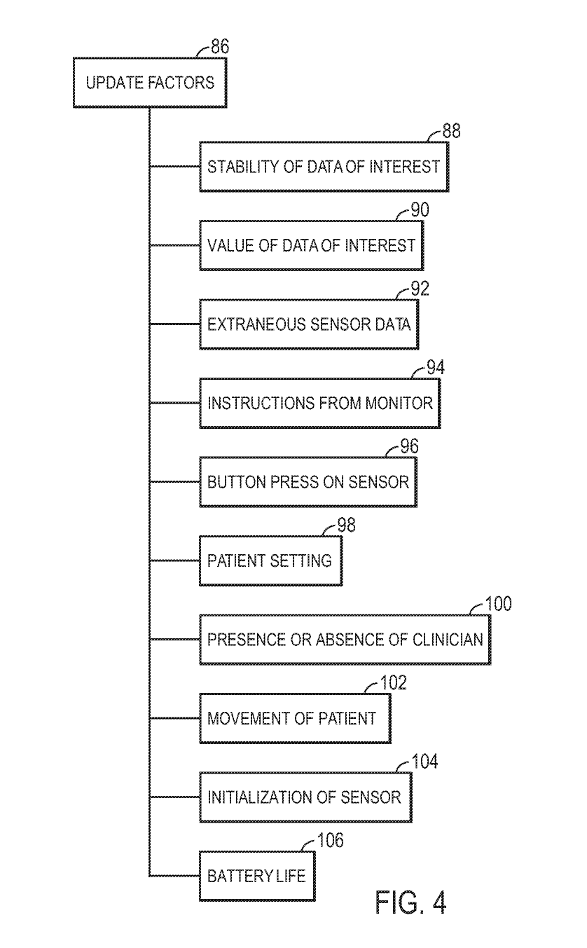

To conserve the amount of power used by the sensor 14, the microprocessor 38 may vary the update rate at which data is transferred using the wireless module 18 to the patient monitor 12 using a variety of techniques, as described in greater detail below. The microprocessor 38 may carry out these techniques based on instructions stored in the RAM 42, the ROM 44, the nonvolatile memory 66, or based on instructions received from the patient monitor 12. Specifically, because the wireless module 18 may consume a substantial amount of power at times when a radio in the wireless module 18 is activated, the radio of the wireless module 18 may generally remain deactivated until data is to be transmitted. The microprocessor 38 may determine a portion of the total raw data that is obtained by the sensor 14 to be transmitted, as well as the specific times at which the portion of the data may be transmitted. During these times, the wireless module 18 may be temporarily activated. Because the wireless module 18 may only be in use at these specific times, less power may be consumed and the life of the battery 70 may be extended. In selecting which of the raw data to transmit and at which times, the microprocessor 38 may consider a variety of factors, including the significance of raw data currently being obtained from the patient 36 by the wireless medical sensor 14. These various factors are described in greater detail below with reference to FIG. 4.

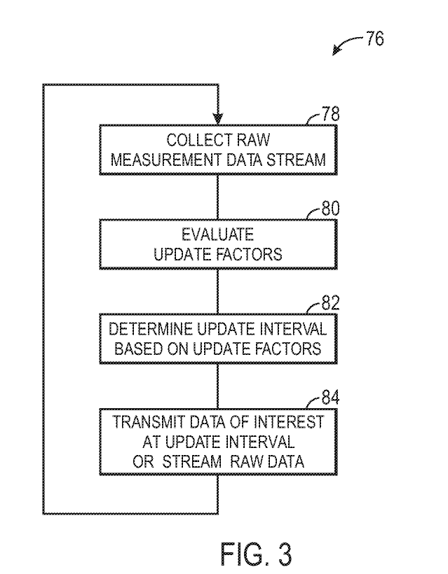

FIG. 3 is a flowchart describing an embodiment of a method for efficiently selecting and transmitting wireless data from the sensor 14 to the patient monitor 12. The method described by the flowchart 74 may enable determination and transmission of a medically sufficient amount of data. Thus, the amount of data sent by the sensor 14 may be reduced as compared to simply transmitting all collected raw data. Accordingly, the amount of power consumed by the wireless module 18 may be reduced. Generally, the sensor 14 may transmit only certain data of interest (e.g., pulse rate, respiration rate, blood oxygen saturation, patient temperature, etc.) at determined intervals to the patient monitor 12, rather than transmit a raw data stream. Based on various update factors, described below, the sensor 14 may increase or decrease the interval at which the data of interest are transmitted to the patient monitor 12.

In a first step 76 of the flowchart 74, the sensor 14 may receive a raw measurement stream, which may be processed by the microprocessor 38. In certain embodiments, the sensor 14 may be a photoplethysmographic sensor configured to obtain a raw 16-bit digital stream of photoplethysmographic data sampled at between approximately 50 Hz or less to 2000 Hz or more (e.g., approximately 1211 Hz). After the data is sampled down to between approximately 10 Hz to 200 Hz (e.g., approximately 57.5 Hz), the microprocessor 38 may further parse the raw stream of data into discrete, meaningful points of data. For example, the microprocessor 38 may break a raw photoplethysmographic data stream into pulse rate, respiration rate data, blood oxygen saturation data, etc. Such discrete data may represent data of interest to be sent to the patient monitor 12, or may be used as update factors in step 78.

In step 78, the microprocessor 38 of the sensor 14 may evaluate one or more update factors, which may represent various criteria for determining an appropriate quantity and rate of data to send to the patient monitor 12. Any number of suitable update factors may considered, many of which may be described with reference to FIG. 4 below. By way of example, in one embodiment, the microprocessor 38 may consider whether the data of interest (e.g., pulse rate, respiration rate, blood oxygen saturation, patient temperature, etc.) has remained stable over a recent historical period (e.g., 5 minutes) or whether any of the data of interest has changed beyond a predetermined threshold.

In step 80, based on the evaluation of the update factors, the microprocessor 38 may determine an appropriate update interval at which to transmit the data of interest. The update interval may be relatively long if the update factors indicate that additional data would be largely superfluous, as may be the case if the patient 36 is very stable. By contrast, the update interval may be relatively short if the update factors indicate that additional data would be medically significant, as may be the case if the patient 36 experiences a rapid change, such as significantly increased or decreased pulse rate, respiration rate. In certain cases, the update interval may be determined to be so short that, rather than transmit only the data of interest to the patient monitor 12, all raw data should be transmitted. The update interval may be any amount of time suitable to provide medically sufficient data to the patient monitor 12 as determined by the wireless medical sensor 14, such as zero seconds (e.g., send raw data stream or a continuous stream of processed values) or periodically every 1 second, every few seconds, minutes, or hours as appropriate to the application. For example, the update interval may be approximately every 1 second, 2 seconds, 5 seconds, 10 seconds, 30 seconds, 1 minute, 2 minutes, 5 minutes, 10 minutes, 30 minutes, 1 hour, 2 hours, 5 hours, etc.

In step 82, the microprocessor 38 may determine whether an amount of time equal to or greater than the determined update interval has passed since the data point of interest was last transmitted to the patient monitor 12. If so, the microprocessor 38 may determine current values of the data of interest, which may then be transmitted wirelessly to the patient monitor 12. Because the radio of the wireless module 18 may be activated only to transmit the data of interest at each update interval the wireless module 18 may consume significantly less power when the update interval is comparatively long. In certain cases, if the update interval is determined to fall beneath a predetermined threshold (e.g., less than one second), the microprocessor 38 may instruct the wireless module 18 to transmit the stream of raw digital data for a predetermined period of time. Following step 82, the process may return to step 76 and may repeat indefinitely.

As described above, the microprocessor 38 of the sensor 14 may evaluate a number of factors to determine the update interval. FIG. 4 represents a schematic diagram 84 of many such update factors 86. As should be appreciated, precisely which update factors 86 may be considered by the microprocessor 38 may be predetermined or may be selected by the microprocessor 38 based on the current condition of the patient 36 and/or the particular medical application for which the sensor 14 is being used.

One factor 88 of the update factors 86 may be the stability of the data of interest obtained by the sensor 14 for a recent historical period. As noted above, the sensor 14 may extract the data of interest (e.g., pulse rate, blood oxygen saturation, etc.) from a raw stream of data (e.g., a raw 16-bit digital stream of photoplethysmographic data sampled at between approximately 50 Hz or less to 2000 Hz or more (e.g., approximately 1211 Hz)). If the data of interest is within a predetermined variability threshold over a recent historical period (e.g., 5 minutes), the factor 88 may weigh in favor of a relatively longer update interval. If the data of interest varies beyond the predetermined variability threshold, the factor 88 may weigh in favor of a relatively shorter update interval. The factor 88 may trigger an immediate update when the data of interest is outside the expected variability, such as if a patient's heart rate suddenly changes from a range of 70-75 bpm to 120 bpm. In determining the update interval based at least in part on the factor 88, the microprocessor 38 may further consider how much the data of interest has varied. For example, the greater the variability of the data of interest, the more the factor 88 may weigh in favor of a shorter update interval.

A second factor 90 of the update factors 86 may be an absolute value of the data of interest obtained by the sensor 14. If the data of interest is within a predetermined acceptable range of values, the factor 90 may weigh in favor of a comparatively longer update interval If the data of interest is higher or lower than the predetermined acceptable range of values, the factor 90 may weigh in favor of a comparatively shorter update interval. By way of example, if the data of interest includes a respiration rate, a predetermined acceptable range of values for an adult patient may be a range of 12 to 20 breaths per minute. A respiration rate less than 12 breaths per minute or greater than 20 breaths per minute may be evaluated by the microprocessor 38 as weighing in favor of a shorter update interval. In determining the update interval based at least in part on the factor 90, the microprocessor 38 may further consider how much the absolute value of the data of interest varies beyond the predetermined acceptable range. For example, the more the data of interest varies from the predetermined acceptable range, the more the factor 90 may weigh in favor of a shorter update interval.

A third factor 92 of the update factors 86 may be the stability of extraneous sensor data or an absolute value of the extraneous sensor data. Extraneous sensor data may represent data not generally being transmitted as data of interest. By way of example, a current patient temperature may be extraneous sensor data when the data of interest is obtained from a photoplethysmographic measurement (e.g., pulse rate, blood oxygen saturation, etc.). Such extraneous sensor data may be obtained, for example, from an extraneous sensor 32 in the wireless medical sensor 14. Like the factors 88 and/or 90, if the extraneous sensor data exceeds a predetermined acceptable range of variability over a recent historical period, or if an absolute value of the extraneous sensor data exceeds a predetermined acceptable range of values, the factor 92 may weigh in favor of a shorter update interval. Similarly, if the extraneous sensor data remains within the predetermined acceptable range of variability over the recent historical period, or if the absolute value of the extraneous sensor data does not exceed the predetermined acceptable range of values, the factor 92 may weigh in favor of a longer update interval. By way of example, if the current patient temperature falls outside a predetermined acceptable range of values (e.g., a range of between 97.6° F. and 99.6° F.), the microprocessor 38 may interpret the factor 92 as weighing in favor of a shorter update interval for photoplethysmographic data of interest. Also like the factors 88 and/or 90, in determining the update interval based at least in part on the factor 92, the microprocessor 38 may further consider how much the extraneous sensor data has varied over time or how much the absolute value of the extraneous sensor data varies beyond the predetermined acceptable range. For example, the more the extraneous sensor data exceeds the predetermined acceptable range, the more the factor 92 may weigh in favor of a shorter update interval.

Express instructions received by the wireless medical sensor 14 from the patient monitor 12 may constitute a fourth factor 94 of the update factors 86. As described below with reference to FIG. 5, in the course of wireless communication with the sensor 14, the patient monitor 12 may transmit updates to sensor parameters in an acknowledgement, or ACK, packet. These sensor parameter updates from the patient monitor 12 may instruct the sensor 14 to send data at a particular interval, to send data in a continuous stream of raw data, or may provide other indications, such as a button press on the monitor 12, which may be interrupted by the sensor 14 and used to determine the update interval. Certain parameters that may govern the operation of the wireless medical sensor 14 or that may weigh in favor of a shorter or longer update interval are described in greater detail below with reference to FIG. 7 To provide one example, by pressing a button on the patient monitor 12, medical personnel may cause the patient monitor 12 to instruct the wireless medical sensor 14 to transmit the raw stream of data.

A fifth factor 96 of the update factors 86 may be a press of the button or switch 34 on the wireless medical sensor 14. If the button or switch 34 is a single button and the button is pressed, the factor 96 may weigh in favor of a shorter update interval. Similarly, if the button or switch 34 is a switch with two or more settings (e.g., low, medium, high, etc.), the setting to which the button or switch 34 has been moved may correspondingly weigh in favor of shorter or longer update intervals, as appropriate. For example, because pressing the button or switch 34 may cause the factor 96 to weigh in favor of a shorter update interval pressing the button or switch 34 may result in the transmission of the raw data stream from the sensor 14 to the patient monitor.

A sixth factor 98 of the update factors 86 may be the current location of the patient 36, which may be supplied to the wireless medical sensor 14 via parameter updates from the patient monitor 12. Because the amount of data from the wireless medical sensor 14 that should be supplied to the patient monitor 12 may vary depending on whether the patient 36 is in surgery, in recovery, or undergoing other tests, the current location of the patient 36 may be considered as one of the update factors 86. Thus, if the patient 36 is currently located in a medical facility room where the patient 36 should be kept under especially close scrutiny, such as an operating room, the factor 98 may weigh in favor of a correspondingly shorter update interval. If the patient 36 is currently located in a medical facility room where the patient 36 may be kept under less scrutiny, such as a recovery room, the factor 98 may weigh in favor of a longer update interval. In determining the update interval based at least in part on the factor 98, the microprocessor 38 may give different locations different weights in favor of a shorter or longer update interval. For example, if the current location is a testing room, such as a CT room, or an operating room, the factor 98 may weigh in favor of a comparatively shorter update interval. However, the factor 98 may weigh more heavily in favor of a shorter update interval if the current location of the patient 36 is the operating room. Similarly, the sensor 14 may be instructed to stop transmitting data or use a very long update internal if the patient 36 is located in close proximity to an instrument which is sensitive to wireless interference. In such a case, if the sensor 14 includes frequency hopping capabilities, the sensor 14 may select an alternate frequency or channel which does not interfere with nearby equipment or sensors located on other patients. In this way, data from a critically ill patient or patient in the operating room may be prioritized higher than patients who are relatively stable.

A seventh factor 100 of the update factors 86 may be the presence or the absence of a clinician proximate to the patient 36, which may be supplied to the wireless medical sensor 14 via parameter updates from the patient monitor 12. For example, if a clinician enters a room where the patient 36 is currently located, the factor 100 may weigh in favor of a comparatively shorter update interval. If the clinician exits the room, the factor 100 may weigh in favor of a comparatively longer update interval. In determining the update interval based at least in part on the factor 100, the microprocessor 38 may weigh the factor 100 more heavily in favor of a shorter or longer update interval based on the number or patient assignment of clinicians present. For example, if a clinician that is not assigned to the patient 36 enters a room where the patient 36 is currently located, the factor 100 may not weigh as heavily in favor of a shorter update interval as when a clinician that is assigned to the patient 36 enters the room.

An eighth factor 102 of the update factors 86 may be the movement of the patient 36, which may be indicated to the wireless medical sensor 14 via parameter updates from the patient monitor 12 or via the movement sensor 74. If the patient 36 is currently moving, indicating that the patient 36 is not at rest or is being moved from one room to another, the factor 102 may weigh in favor of a comparatively shorter update interval. If the patient 36 is not currently moving, the factor 102 may weigh in favor of a comparatively longer update interval. Additionally, the amount of current patient movement may further affect the weight of the factor 102 in favor of a comparatively shorter or longer update interval. In another example, transmission of the heart rate of the patient 36 may be suppressed if an accelerometer of the movement sensor 74 detects excessive motion artifact and the calculated heart rate is less likely to be accurate than a previous value.

A ninth factor 104 of the update factors 86 may be an initialization status of the sensor 14. For a predetermined period of time while the sensor is being initialized (e.g., 5 minutes), the update rate of the sensor 14 may be temporarily increased dramatically, such that the raw data stream is supplied to the patient monitor 12. By supplying a raw data stream during the initialization of the sensor 14, a clinician for other medical personnel may properly fit the sensor 14 to the patient 36. In this way, the factor 104 may weigh very heavily in favor of a shorter update interval when the sensor 14 has recently been activated.

A tenth factor 106 of the update factors 86 may be a battery life of the wireless medical sensor 14. If the battery 70 of the sensor 14 has more than a predetermined amount of remaining battery life, the factor 106 may weigh in favor of a comparatively shorter update interval. If the battery 70 has less than the predetermined amount of remaining battery life, the factor 106 may weigh in favor of a comparatively longer update interval. This factor 106 may also account for the transmit power required to send error-free data at the last update. For instance, when the patient 36 is relatively far from the receiver, more transmit power may be required, so less frequent updates may take place, especially at lower battery 70 reserves.

FIG. 5 is a schematic communication diagram 108 describing communication between the wireless medical sensor 14 and the patient monitor 12. As shown in the communication diagram 108, communication between the wireless medical sensor 14 and the patient monitor 12 may begin once the sensor 14 has obtained 110 the raw data stream and has evaluated 112 the one or more update factors 86. Having determined the update interval based on the evaluation 112 of the update factors 86, the sensor 14 may begin the process of transmitting the data of interest at the start of the next update interval

Transmission of the data of interest from the sensor 14 to the patient monitor 12 may begin at the start of an update interval when the sensor 14 activates 114 a radio of the wireless module 18. The sensor 14 may concurrently or subsequently sample 116 the current data of interest (e.g., pulse rate, blood oxygen saturation, etc.) from the raw data stream (e.g., a raw 16-bit digital stream of photoplethysmographic data sampled at 100 Hz). The sampled data of interest may be a much smaller quantity of data than the raw data stream, and may be, for example, a single 8-bit value. Additionally or alternatively, the sensor 14 may sample 116 the current data of interest from the raw data stream, optionally process the data, and packetize the data for transmission prior to powering up the radio of the wireless module 18. Doing so may minimize the amount of time the radio of the wireless module 18 is active.

Thereafter, the wireless medical sensor 14 may wirelessly transmit 118 the data of interest to the patient monitor 12. In addition to the data of interest, the sensor 14 may also transmit 118 other information regarding the sensor 14 status, such as remaining battery life. If reliable delivery is needed, the patient monitor 12 may reply 120 with a wireless acknowledgment packet, or ACK, which may also include one or more sensor parameter updates. The data contained in the parameter update of the ACK packet may instruct the sensor 14 to operate in a particular way, or may convey information regarding the update factors 86, as described above. Including the information part of the ACK packet may generally mean that the sensor 14 does not have to power a receiver of the wireless module 18 at other times.

Following the transmission 118 of the data of interest and optional reply 120 from the patient monitor 12, the sensor 14 may deactivate 122 the radio of the wireless module 18. Depending on the selected protocol, the sensor 14 may power up the transmitter of the wireless module 18 one more time to ACK any new instructions from the patient monitor 12. For the remainder of the update interval, the wireless module 18 may consume only a minimal amount of power. Because the wireless module 18 does not continually consume power, the battery 70 of the sensor 14 may provide power for a longer amount of time or may be smaller than those of comparable sensors that do not perform the techniques disclosed herein. Until circumstances change, and the update factors indicate a different update interval, the data of interest may continue to be transmitted at the update interval, which may start again when the radio of the wireless module is again activated 124.

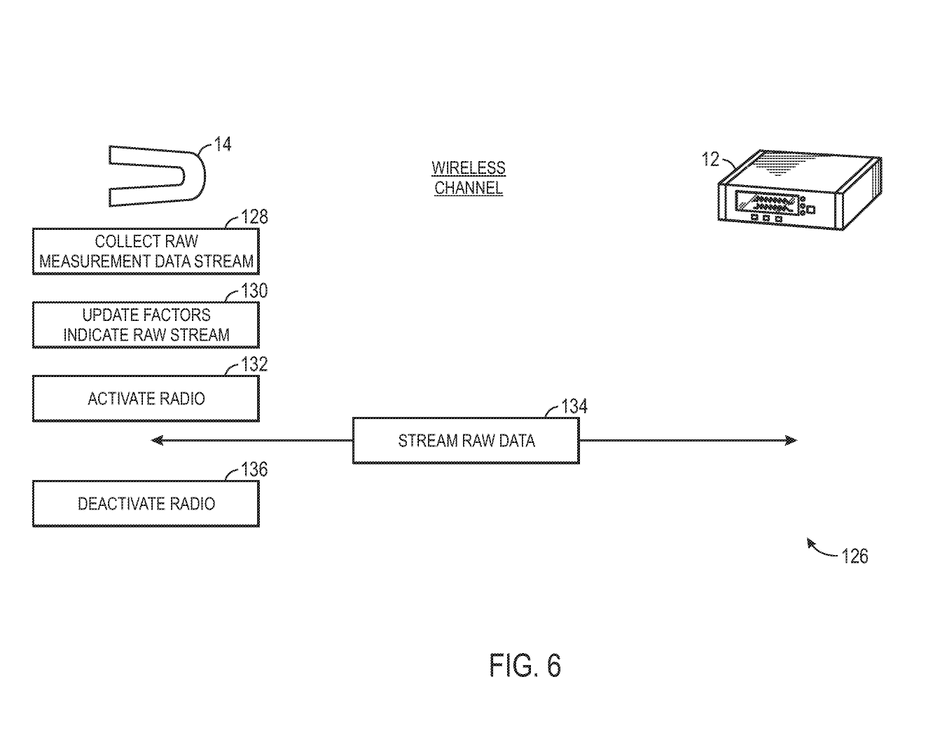

FIG. 6 is another schematic communication diagram 126 describing communication between the wireless medical sensor 14 and the patient monitor 12, which may take place when the update factors 86 indicate that the raw data stream should be transmitted in its entirety. As shown in the communication diagram 126, communication between the wireless medical sensor 14 and the patient monitor 12 may begin once the sensor 14 has obtained 128 the raw data stream and has evaluated 130 the one or more update factors 86. Having determined that the update interval based on the evaluation 130 indicates that the raw data stream should be transmitted, the sensor 14 may begin the process of transmitting the raw data stream without waiting for the start of an update interval.

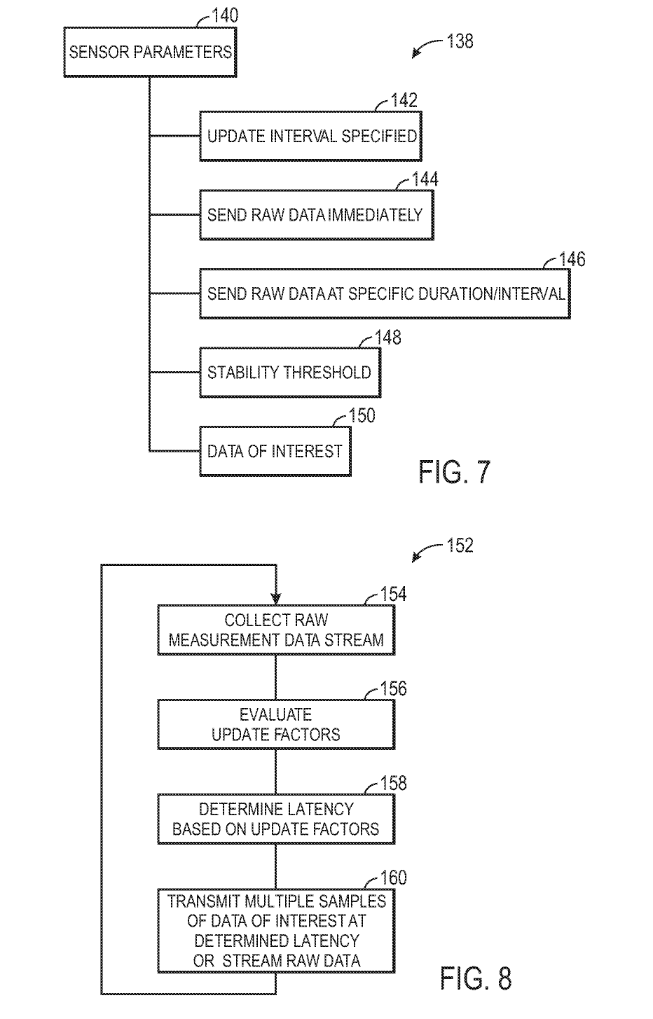

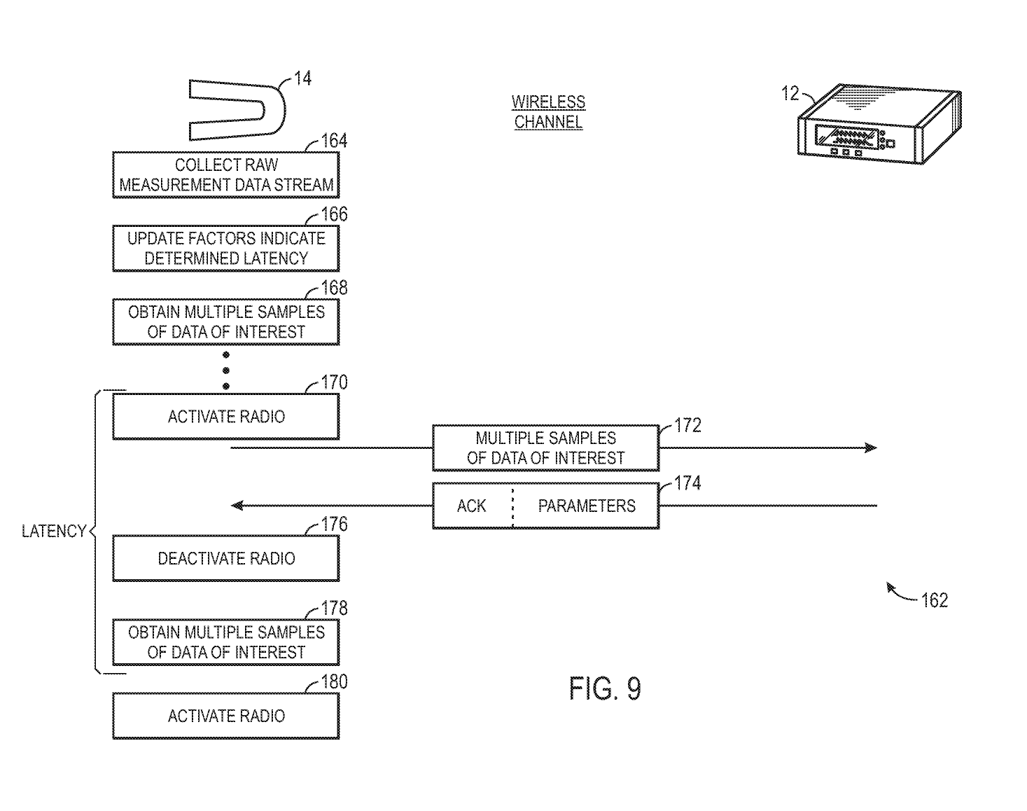

Transmission of the raw data stream from the sensor 14 to the patient monitor 12 may begin when the sensor 14 activates 132 a radio of the wireless module 18. Thereafter, the wireless medical sensor 14 may wirelessly stream 134 the raw data to the patient monitor 12. Communication during the streaming 134 of the raw data may include various replies from the patient monitor 12. After a predetermined time, the sensor 14 may deactivate 136 the radio of the wireless module 18, and the process may repeat until circumstances change and the update interval is increased. As noted below with reference to FIGS. 8 and 9, if latency can be tolerated, it may be more efficient to queue several raw samples and power up the radio of the wireless module 18 only periodically. For example, the sensor 12 may queue 100 ms to 1 minute of raw data before powering on the radio of the wireless module 18 for only a few hundred milliseconds to transmit the data.

As described above, the operation of the wireless medical sensor 14 may be governed by various sensor parameters. These sensor parameters may be occasionally updated by the patient monitor 12 via parameter updates in an acknowledgement packet, or ACK, as described above with reference to FIG. 5. FIG. 7 is a diagram 138 that describes many such sensor parameters 140.

A first parameter 142 of the sensor parameters 140 may be a specified update interval. The parameter 142 may predetermine the update interval at which the sensor 14 transmits the data of interest to the patient monitor 12. If the parameter 142 sets a specific update interval, the parameter 142 may override the update interval determination that the sensor 14 may generally undertake, and the sensor 14 may employ the specified update interval.

A second parameter 144 of the sensor parameters 140 may be an indication to the sensor 14 that the raw data stream should be transmitted to the patient monitor 12 immediately. By way of example, a clinician may press a button on the patient monitor 12, and the patient monitor 12 may indicate via parameter updates in the next ACK packet that the raw data stream is desired. Thus, upon receiving parameter updates with such an update to the parameter 144, the wireless medical sensor 14 may begin to transmit the raw data stream to the patient monitor 12.

A third parameter 146 of the sensor parameters 140 may be a specification that raw data should be sent at specific predetermined intervals and for specific durations. For example, the parameter 146 may specify that the raw data stream is to be sent every hour for one minute. Thus, the parameter 146 may instruct the sensor 14 to supplement the data of interest with the raw data.

A fourth parameter 148 of the sensor parameters 140 may be a specification of the predetermined variability threshold or the predetermined range of acceptable values, as may be employed by the update factors 88-92. A fifth parameter 150 of the sensor parameters 140 may be a specification of the data of interest. For example, the parameter 150 may specify that the data of interest is pulse rate and/or blood oxygen saturation when the raw data is a photoplethysmographic data stream.

The sensor parameters 140 illustrated in the diagram 138 are intended to be exemplary and not exclusive. As such, it should be understood that the sensor parameters 140 may further include other data that may enable the wireless medical sensor 14 to effectively carry out the techniques disclosed herein. For example, the sensor parameters 140 may also include other data that indicate, for example, a current patient location or a current clinician location, which may be employed to weigh various update factors 86.

FIG. 8 is a flowchart 152 of another embodiment of a method for efficiently selecting and transmitting wireless data from the sensor 14 to the patient monitor 12. The method described by the flowchart 152 may enable determination and transmission of a medically sufficient amount of information by sampling the data of interest (e.g., pulse rate, respiration rate, blood oxygen saturation, patient temperature, etc.) at a sampling interval and thereafter transmitting the sampled data at a determined latency. Thus, the amount of data sent by the sensor 14 may be reduced, particularly as compared to simply transmitting all collected raw data, and the amount of power consumed by the wireless module 18 may be correspondingly reduced. Based on the various update factors, described in greater detail above, the sensor 14 may increase or decrease the sampling interval and/or latency that the data of interest are transmitted to the patient monitor 12.

In a first step 154 of the flowchart 156, the sensor 14 may receive a raw measurement stream, which may be processed by the microprocessor 38. For example, in certain embodiments, the sensor 14 may be a photoplethysmographic sensor configured to obtain a raw 16-bit digital stream of photoplethysmographic data sampled at between approximately 50 Hz or less to 2000 Hz or more (e.g., approximately 1211 Hz). After the data is sampled down to between approximately 10 Hz to 200 Hz (e.g., approximately 57.5 Hz), the microprocessor 38 may further parse the raw stream of data into discrete, meaningful points of data. For example, the microprocessor 38 may break a raw photoplethysmographic data stream into pulse rate data, blood oxygen saturation data, etc. Such discrete data may represent data of interest to be sent to the patient monitor 12 or data for use in evaluating the update factors 86 in step 156.

In step 156, the microprocessor 38 of the sensor 14 may evaluate one or more update factors 86, which may represent various criteria for determining an appropriate quantity and rate of data to send to the patient monitor 12. Any number of suitable update factors may considered, many of which may be described with reference to FIG. 4 above. By way of example, in one embodiment, the microprocessor 38 may consider whether the data of interest (e.g., pulse rate, respiration rate, blood oxygen saturation, patient temperature, etc.) has remained stable over a recent historical period (e.g., 5 minutes) or whether any of the data of interest has changed beyond a predetermined threshold.

In step 158, based on the evaluation of the update factors 86 of step 156, the microprocessor 38 may determine an appropriate sampling rate and latency at which to transmit the data of interest. The sampling rate and/or the latency may be relatively fast if the update factors 86 indicate that additional data would be medically significant, as may be the case if the patient 36 experiences a rapid change, such as significantly increased or decreased pulse rate, respiration rate, etc. By contrast, the sampling rate and/or the latency may be relatively slow if the update factors 86 indicate that additional data would be largely superfluous, as may be the case if the patient 36 is very stable. In certain cases, the sampling rate and/or the latency may be determined to be so fast that, rather than transmit only the data of interest to the patient monitor 12 at a given latency, all raw data should be transmitted immediately. The latency may be similar to the update interval, in that the latency may include any amount of time suitable to provide medically sufficient data to the patient monitor 12 as determined by the wireless medical sensor 14, such as zero seconds (e.g., send raw data stream or a continuous stream of processed values) or periodically every 1 second, every few seconds, minutes, or hours as appropriate to the application. By way of example, the latency may be 1 second, 2 seconds, 5 seconds, 10 seconds, 30 seconds, 1 minute, 2 minutes, 5 minutes, 10 minutes, 30 minutes, 1 hour, 2 hours, 5 hours, etc.

In step 160, the microprocessor 38 may continually sample the data of interest at the sampling rate determined in step 158. The sampled data may be stored in the RAM 42 or nonvolatile memory 66. The microprocessor 38 may also determine whether an amount of time equal to or greater than the determined latency has passed since the sampled data of interest were last transmitted to the patient monitor 12. If so, the microprocessor 38 may cause the sampled data of interest stored in the RAM 42 or the nonvolatile memory 66 to be transmitted wirelessly to the patient monitor 12. Because the radio of the wireless module 18 may be activated only to transmit the sampled data of interest at the determined latency, the wireless module 18 may consume significantly less power when the latency is comparatively long. In certain cases, if the latency is determined to fall beneath a predetermined threshold (e.g., less than one second), the microprocessor 38 may instruct the wireless module 18 to transmit the stream of raw digital data for a predetermined period of time. Following step 160, the process may return to step 154 and may repeat indefinitely.

FIG. 9 is another schematic communication diagram 162 describing communication between the wireless medical sensor 14 and the patient monitor 12. The communication described by the flowchart 162 may describe determination and transmission of a medically sufficient amount of data by sampling the data of interest (e.g., pulse rate, respiration rate, blood oxygen saturation, patient temperature, etc.) at a sampling interval and transmitting the sampled data at a determined latency. As shown in the communication diagram 162, communication between the wireless medical sensor 14 and the patient monitor 12 may begin once the sensor 14 has obtained 164 the raw data stream and has evaluated 166 the one or more update factors 86. Having determined the sampling rate and/or the latency based on the evaluation 166 of the update factors 86, the sensor 14 may obtain multiple samples 168 of the data of interest at the determined sampling rate until the start of the next latency interval.

Transmission of the data of interest from the sensor 14 to the patient monitor 12 may begin when the determined latency has been reached and the sensor 14 activates 170 a radio of the wireless module 18. The sensor 14 may wirelessly transmit 172 the multiple samples of the data of interest to the patient monitor 12. In addition to the data of interest, the sensor 14 may also transmit 172 other information regarding the sensor 14 status, such as remaining battery life. The patient monitor 12 may reply 174 with a wireless acknowledgment packet, or ACK, which may also include one or more sensor parameter updates. The data contained in the parameter update of the ACK packet may instruct the sensor 14 to operate in a particular way, or may convey information regarding the update factors 86, as described above.

Following the transmission 172 of the multiple samples of the data of interest and the reply 174 from the patient monitor 12, the sensor 14 may deactivate 176 the radio of the wireless module 18. For the remainder of the latency interval the wireless module 18 may consume only a minimal amount of power and the microprocessor 38 may continue to evaluate the update factors 86 and obtain multiple samples 178 of the data of interest. Because the wireless module 18 does not continually consume power, the battery 70 of the sensor 14 may provide power for a longer amount of time or may be smaller than those of comparable sensors that do not perform the techniques disclosed herein. Until circumstances change, and the update factors 86 indicate a different sampling rate and/or latency, the multiple samples of the data of interest may continue to be transmitted at the latency, which may start again when the radio of the wireless module is again activated 180

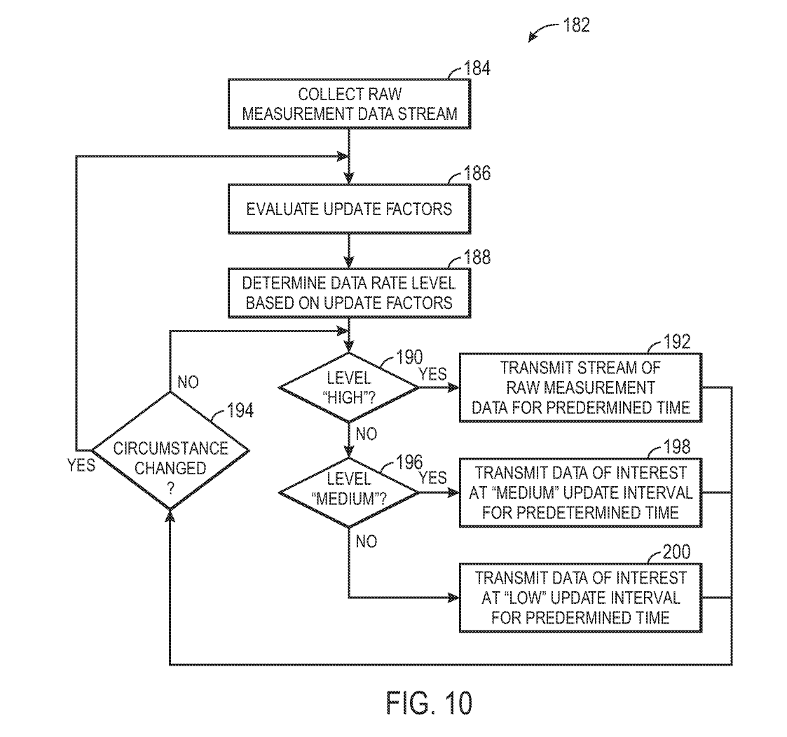

FIG. 10 depicts a flowchart 182 describing an embodiment of a method for transmitting data at discrete levels. In a first step 184, the sensor 14 may collect the stream of raw measurement data from the patient 36. In a step 186, various factors, such as the update factors 86 described above with reference to FIG. 4, may be evaluated by the sensor 14. Based on the factors evaluated in step 186, the sensor 14 may determine a discrete data rate transmission level in step 188. In the embodiment of the flowchart 182, the sensor 14 may select between three predetermined discrete data rate levels of “low,” “medium,” and “high.” Any suitable number of discrete data rate levels may be defined, and the number of discrete data rate levels may vary depending on the various update factors 86 considered in step 186.

In a subsequent decision 190, if the discrete data rate level is “high,” the sensor 14 may, in step 192, transmit the stream of raw measurement to the patient monitor 12 for a predetermined time. After the predetermined time has passed, step 192 may end and the process may flow to a decision 194, at which the sensor 14 may evaluate whether circumstances informing the update factors 86 have changed. If circumstances remain the same, the process may return to the decision 190, where, because the data rate level remains set to “high,” the process may return to step 192.

Returning to the decision 194, if circumstances have changed, the update factors 86 may be evaluated again in step 186, and a new data rate level may be determined in step 188. If the data rate level is not “high,” as determined in the decision 190, the process may flow to a decision 196. If the data rate level is “medium,” the sensor 14 may transmit a sample of the data of interest at a medium update interval for a predetermined time. By way of example, the sensor 14 may transmit pulse rate measurements once every five seconds for one minute. After the predetermined time has passed, the process may flow to the decision 194 for reevaluation of circumstances.

Returning to the decision block 196, if the data rate level is not “medium,” and thus, is “low,” step 200 may take place. In step 200, the sensor 14 may transmit the data of interest at a low update rate for a predetermined period of time. For example, the sensor 14 may transmit pulse rate data once every thirty seconds for 5 minutes. Following the predetermined time, the circumstances may be reevaluated in the decision 194.

While the embodiments set forth in the present disclosure may be susceptible to various modifications and alternative forms, specific embodiments have been shown by way of example in the drawings and have been described in detail herein. However, it should be understood that the disclosure is not intended to be limited to the particular forms disclosed. The disclosure is to cover all modifications, equivalents, and alternatives falling within the spirit and scope of the disclosure as defined by the following appended claims.

What is claimed is:

1. A medical device comprising:
a medical sensor capable of obtaining a raw measurement from a patient;
data processing circuitry capable of sampling the raw measurement to obtain a plurality of values and capable of determining an update interval based at least in part on an update factor associated with a status of the patient; and
wireless transmission circuitry capable of wirelessly transmitting one of the plurality of values to an external wireless receiver at the update interval.

2. The device of claim 1, wherein the medical sensor comprises a photoplethysmographic sensor; a respirator band; a temperature sensor; a blood pressure sensor; an ECG sensor; or a pulse transit time sensor; or any combination thereof.

3. The device of claim 1, wherein the data processing circuitry is capable of determining the update interval based at least in part on the update factor associated with the status of the patient, wherein the update factor comprises a historical stability of the plurality of values; an absolute value of one of the plurality of values; a historical stability of a plurality of extraneous sensor values obtained from an extraneous medical sensor; an absolute value of an extraneous sensor value obtained from the extraneous medical sensor; an instruction from an external device; a button press or switch setting on the medical device; a current location of the patient; a current location of a clinician; a movement of the patient; an initialization status of the medical device; or a remaining battery life of the medical device; or any combination thereof.

4. The device of claim 1, comprising a button, wherein the wireless transmission circuitry is capable of transmitting the raw measurement to the external wireless receiver when the button is pressed.

5. The device of claim 1, comprising another medical sensor capable of obtaining another raw measurement of the patient, wherein the data processing circuitry is capable of sampling the other raw measurement to obtain a plurality of other values, wherein the data processing circuitry is capable of determining the update interval based at least in part on the update factor, and wherein the update factor comprises a historical stability of the plurality of other values or an absolute value of one of the plurality of extraneous sensor values.

6. The device of claim 1, wherein the wireless transmission circuitry is capable of transmitting the raw measurement to the external wireless receiver for a period of time when the determined update interval is beneath a threshold.

7. The device of claim 1, wherein the wireless transmission circuitry is capable transmitting a portion of the plurality of values to the external wireless receiver at the update interval.

8. The device of claim 1, comprising a memory device capable of storing at least a portion of the plurality of values, wherein the portion of the plurality of values have been obtained at an approximately constant sampling rate.

9. A method comprising:
obtaining, using a medical sensor, a raw measurement from a patient;
determining, using a processor, an update interval based at least in part on at least one update factor associated with a status of the patient; and
transmitting, using a wireless radio physically coupled to the medical sensor, a value obtained from the raw measurement to an external wireless receiver at the update interval.

10. The method of claim 9, wherein the obtained raw measurement comprises a pulse rate; a blood pressure saturation; a measure of total hemoglobin; a respiration rate; a temperature; an ECG; a blood pressure; or a pulse transit time; or any combination thereof.

11. The method of claim 9, wherein determining the update interval based at least in part on the at least one update factor associated with a status of the patient comprises evaluating the at least one update factor, wherein the at least one update factor comprises a historical stability of a plurality of values obtained from the raw measurement; an absolute value of one of the plurality of values; a historical stability of a plurality of extraneous sensor values obtained from an extraneous medical sensor; an absolute value of an extraneous sensor value obtained from the extraneous medical sensor; an instruction from an external device; a selection of a button or switch physically coupled to medical sensor; a current location of the patient; a current location of a clinician; a movement of the patient; an initialization status of the medical sensor; or a remaining battery life of a battery physically coupled to the medical sensor; or any combination thereof.

12. The method of claim 9, wherein the value is transmitted at the update interval only when the update interval is above a threshold.

13. The method of claim 12, comprising transmitting the raw measurement when the update interval is below a threshold.

14. The method of claim 9, comprising receiving an acknowledgement from the external wireless receiver, wherein the acknowledgement comprises data associated with the status of the patient.

15. A system comprising:
an electronic patient monitor capable of wirelessly receiving a measurement of a patient; and
a wireless medical sensor capable of obtaining a raw measurement from the patient, determining an update interval based at least in part on an update factor associated with a status of the patient, and wirelessly transmitting a value obtained from the raw measurement to the electronic patient monitor at the update interval.

16. The system of claim 15, wherein the electronic patient monitor is capable of wirelessly transmitting instructions to the wireless medical sensor.

17. The system of claim 15, wherein the electronic patient monitor is capable of wirelessly transmitting at least one sensor operating parameter to the wireless medical sensor.

18. The system of claim 15, wherein the wireless medical sensor is capable of determining the update interval based at least in part on a sensor operating parameter, wherein the operating parameter comprises a specified update interval; a specified indication that the raw data is to be transmitted immediately; a specified variability threshold of values obtained from the raw measurement; a specified range of acceptable values obtained from the raw measurement; a specified type of the value to be obtained from the raw measurement; a current location of the patient; a current location of a clinician; or any combination thereof.

19. The system of claim 15, wherein the wireless medical sensor is capable of wirelessly transmitting the raw measurement at a second update interval.

20. The system of claim 19, wherein the second update interval is defined by a sensor operating parameter.

21. The method of claim 15, wherein the wireless medical sensor is capable of wirelessly transmitting a remaining battery life of the wireless medical sensor to the electronic patient monitor when the value is transmitted.

22. A method comprising:
obtaining, using a medical sensor, a raw measurement from a patient;
determining, using a processor physically coupled to the medical sensor, a data rate level based at least in part on an update factor associated with a status of the patient; and
transmitting, using a wireless radio physically coupled to the medical sensor, a value obtained from the raw measurement to an external wireless receiver at an update interval, wherein the update interval is a value associated with the data rate level.

23. The method of claim 22, wherein determining the data rate level comprises selecting one of a plurality of levels.

24. The method of claim 22, wherein transmitting the value comprises transmitting, one at a time for a period of time, a plurality of values obtained from the raw measurement to the external wireless receiver at the update interval.

25. The method of claim 24, wherein the period of time is a value associated with the data rate level.
